(19) United States
(12) Patent Application Publication
(10) Pub. No.: US 2026/0240941 A1
(43) Pub. Date: Aug. 20, 2026

(54) TREATMENT AND PREVENTION OF AGENT-INDUCED LIVER CONDITIONS OR DISEASES

(71) Applicant: FARSIGHT MEDICAL TECHNOLOGY (SHANGHAI) CO., LTD., Shanghai (CN)

(72) Inventors: Jia XIAO, Qingdao (CN); Ching Pong Mak, Basel (CH); Fashu Ma, Shanghai (CN); Dong Xiao, Shanghai (CN); Kwok Fai So, Hong Kong (CN); Zhaodi Che, Qingdao (CN)

(21) Appl. No.: 19/159,324

(22) PCT Filed: Mar. 1, 2024
(86) PCT No.: PCT/CN2024/079653

§ 371 Date: Aug. 25, 2025

(30) Foreign Application Priority Data
Mar. 3, 2023 (WO) PCT/CN2023/079480

(51) Int. Cl. A61K 38/13; A61P 1/16; A61P 29/00

(52) U.S. Cl. CPC A61K 38/13; A61P 1/16; A61P 29/00

(57) ABSTRACT

Provided herein is Compound 1 or a pharmaceutically acceptable salt thereof for use in the prevention and/or treatment of a liver condition or disease in a subject exposed to an agent capable of inducing the liver condition or disease.

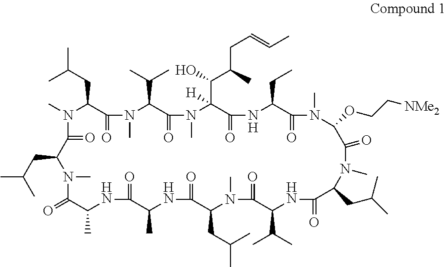 [SMILES: C/C=C/C[C@@H](C)[C@@H](O)[C@H]1C(=O)N[C@@H](CC)C(=O)N(C)[C@H](OCCN(C)C)C(=O)N(C)[C@@H](CC(C)C)C(=O)N[C@@H](C(C)C)C(=O)N(C)[C@@H](CC(C)C)C(=O)N[C@@H](C)C(=O)N[C@H](C)C(=O)N(C)[C@@H](CC(C)C)C(=O)N(C)[C@@H](CC(C)C)C(=O)N(C)[C@@H](C(C)C)C(=O)N1C]

### BACKGROUND OF THE INVENTION

Certain agents, including approved drug substances, are known to induce liver conditions or diseases such as hepatic inflammation, hepatocellular necrosis, hepatic lesions, liver fibrosis, liver cirrhosis, acute liver failure, alcoholic fatty liver, alcoholic steatohepatitis, alcoholic liver disease, and hepatocellular cancer. The agents include common household items such as acetaminophen (APAP) and beverage alcohol. Acetaminophen toxicity is a leading cause of liver transplantation worldwide and responsible for hospitalizations, emergency room visits, and deaths. Alcoholic liver disease is the most common chronic liver disease and a major cause of deaths worldwide. Alcoholic liver disease and alcohol induced liver conditions and diseases such as alcohol induced hepatitis have nearly no treatment options other than abstinence and brief exposure to corticosteroids. Other agents that cause liver injury include small molecule drugs, biological drugs, traditional Chinese medicines, natural medicines, and dietary supplements. Increased usage of herbal and dietary supplements has been linked to an increase in herbal and dietary supplement induced hepatotoxicity.

U.S. Pat. No. 6,583,265 discloses Compound 1:

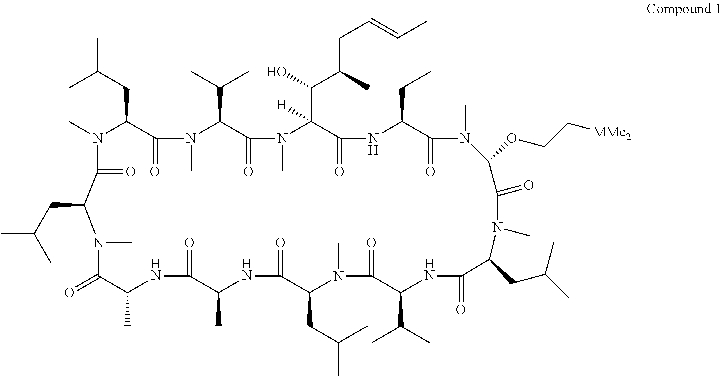

This compound is featured in Example 27 in the patent U.S. Pat. No. 6,583,265, which includes many hundreds of named compounds having modifications at various positions around the ring. However no biological testing data or particular uses are described for this compound or related analogues.

Compound 1 is also disclosed in WO2019/0169572, for use in the treatment or prevention of acute or chronic inflammatory disorders such as acute kidney injury, ischaemia-reperfusion injury, or chronic or acute pancreatitis. Compound 1 is further disclosed in WO2021/068957, for use in the prevention and/or treatment of a kidney condition or disease in a subject induced by a nephrotoxic drug substance, or an endogenous nephrotoxin. WO2019/0169572 and WO2021/068957 do not disclose use of Compound 1 in the prevention and/or treatment of a liver condition or disease in a subject exposed to an agent capable of inducing the liver condition or disease.

It is thus an object of the present disclosure to provide for a novel treatment and prophylaxis of liver conditions or diseases associated with, or induced by exposure to one or more agents, for example, induced by exposure to an alcohol such as a beverage alcohol, and for the protection of the liver in subjects receiving treatment with drug substances.

Further objects of the disclosure will be clear on the basis of the following description, examples, and claims.

### SUMMARY OF THE INVENTION

In a first aspect, the present disclosure relates to Compound 1 or a pharmaceutically acceptable salt thereof

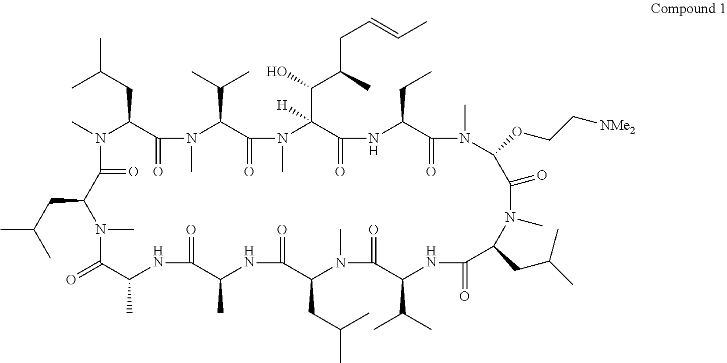 [SMILES: C/C=C/C[C@@H](C)[C@@H](O)[C@H]1C(=O)N[C@@H](CC)C(=O)N(C)[C@H](OCCN(C)C)C(=O)N(C)[C@@H](CC(C)C)C(=O)N[C@@H](C(C)C)C(=O)N(C)[C@@H](CC(C)C)C(=O)N[C@@H](C)C(=O)N[C@H](C)C(=O)N(C)[C@@H](CC(C)C)C(=O)N(C)[C@@H](CC(C)C)C(=O)N(C)[C@@H](C(C)C)C(=O)N1C]

for use in the prevention and/or treatment of a liver condition or disease in a subject exposed to an agent capable of inducing the liver condition or disease. In a further aspect, provided is the use of Compound 1 or a pharmaceutically acceptable salt thereof in the manufacture of a medicament for the prevention and/or treatment of a liver condition or disease in a subject exposed to an agent, i.e., a drug substance or other agent capable of inducing the liver condition or disease. In yet a further aspect, the current disclosure provides for a method for preventing and/or treating a liver condition or disease in a subject exposed to an agent capable of inducing the liver condition or disease, the method comprising administering Compound 1 or a pharmaceutically acceptable salt thereof to said subject. In further aspect, the agent is acetaminophen (APAP) or an alcohol.

### DESCRIPTION OF THE DRAWINGS

FIG. 1 depicts the effect of Compound 1 dosing on Necrotic Area (%) of the liver after 6 hrs administration of acetaminophen (APAP) as described in Example 1. Depicted from left to right on the x-axis are: the Control Group (no administration of APAP, black circles), the Vigilance Group (no administration of APAP, administration of 10 mg/kg Compound 1, black squares), the APAP Group (administration of APAP, black triangles), the Compound 1-1 mg Group (administration of APAP, administration of 1 mg/kg Compound 1, inverted black triangles); the Compound 1-2.5 mg Group (administration of APAP, administration of 2.5 mg/kg Compound 1, open circles); the Compound 1-5 mg Group (administration of APAP, administration of 5 mg/kg Compound 1, open squares); the Compound 1-10 mg Group (administration of APAP, administration of 10 mg/kg Compound 1, open triangles); and the Positive Control Group (administration of APAP, administration of 150 mg/kg N-acetylcysteine “NAC”, open inverted triangles). The 5 mg/kg and 10 mg/kg Compound 1 levels showed liver tissue protection after 6 hours APAP administration.

FIG. 2 depicts the effect of Compound 1 dosing on serum ALT (alanine aminotransferase) and AST (aspartate aminotransferase) biomarker levels of the liver after 6 hrs administration of acetaminophen (APAP) as described in Example 1. Depicted from left to right on the x-axis are: the Control Group (no administration of APAP, black circles), the Vigilance Group (no administration of APAP, administration of 10 mg/kg Compound 1, black squares), the APAP Group (administration of APAP, black triangles), the Compound 1-1 mg Group (administration of APAP, administration of 1 mg/kg Compound 1, inverted black triangles); the Compound 1-2.5 mg Group (administration of APAP, administration of 2.5 mg/kg Compound 1, open circles); the Compound 1-5 mg Group (administration of APAP, administration of 5 mg/kg Compound 1, open squares); the Compound 1-10 mg Group (administration of APAP, administration of 10 mg/kg Compound 1, open triangles); and the Positive Control Group (administration of APAP, administration of 150 mg/kg N-acetylcysteine “NAC”, open inverted triangles) *** refers to P in the range from 0 to 0.001, based on one-way ANOVA; Tukey's multiple comparison test. All Compound 1 levels showed reduced ALT and AST biomarker levels after 6 hours APAP administration.

FIG. 3 depicts the effect of Compound 1 dosing on TNF-α (tumor necrosis factor alpha), IL-1β (interleukin-1 beta), and IL-6 (interleukin-6) inflammation biomarker levels of the liver after 6 hrs administration of acetaminophen (APAP) as described in Example 1. Depicted from left to right on the x-axis are: the Control Group (no administration of APAP, black circles), the Vigilance Group (no administration of APAP, administration of 10 mg/kg Compound 1, black squares), the APAP Group (administration of APAP, black triangles), the Compound 1-1 mg Group (administration of APAP, administration of 1 mg/kg Compound 1, inverted black triangles); the Compound 1-2.5 mg Group (administration of APAP, administration of 2.5 mg/kg Compound 1, open circles); the Compound 1-5 mg Group (administration of APAP, administration of 5 mg/kg Compound 1, open squares); the Compound 1-10 mg Group (administration of APAP, administration of 10 mg/kg Compound 1, open triangles); and the Positive Control Group (administration of APAP, administration of 150 mg/kg N-acetylcysteine “NAC”, open inverted triangles) * refers to P in the range from 0.01 to 0.05, ** refers to P in the range 0.001 to 0.01, and *** refers to P in the range from 0 to 0.001, based on one-way ANOVA; Tukey's multiple comparison test. The 2.5 mg/kg, 5 mg/kg and 10 mg/kg Compound 1 levels showed reduced TNF-α, IL-1β, and/or IL-6 inflammation biomarker levels after 6 hours APAP administration.

FIG. 4 depicts the effect of Compound 1 dosing on Necrotic Area (%) of the liver after 24 hrs administration of acetaminophen (APAP) as described in Example 1. Depicted from left to right on the x-axis are: the Control Group (no administration of APAP, black circles), the Vigilance Group (no administration of APAP, administration of 10 mg/kg Compound 1, black squares), the APAP Group (administration of APAP, black triangles), the Compound 1-1 mg Group (administration of APAP, administration of 1 mg/kg Compound 1, inverted black triangles); the Compound 1-2.5 mg Group (administration of APAP, administration of 2.5 mg/kg Compound 1, open circles); the Compound 1-5 mg Group (administration of APAP, administration of 5 mg/kg Compound 1, open squares); the Compound 1-10 mg Group (administration of APAP, administration of 10 mg/kg Compound 1, open triangles); and the Positive Control Group (administration of APAP, administration of 150 mg/kg N-acetylcysteine “NAC”, open inverted triangles) ** refers to P in the range 0.001 to 0.01, based on one-way ANOVA; Tukey's multiple comparison test. The 5 mg/kg and 10 mg/kg Compound 1 levels showed liver tissue protection after 24 hours APAP administration.

FIG. 5 depicts the effect of Compound 1 dosing on serum ALT (alanine aminotransferase) and AST (aspartate aminotransferase) biomarker levels of the liver after 24 hrs administration of acetaminophen (APAP) as described in Example 1. Depicted from left to right on the x-axis are: the Control Group (no administration of APAP, black circles), the Vigilance Group (no administration of APAP, administration of 10 mg/kg Compound 1, black squares), the APAP Group (administration of APAP, black triangles), the Compound 1-1 mg Group (administration of APAP, administration of 1 mg/kg Compound 1, inverted black triangles); the Compound 1-2.5 mg Group (administration of APAP, administration of 2.5 mg/kg Compound 1, open circles); the Compound 1-5 mg Group (administration of APAP, administration of 5 mg/kg Compound 1, open squares); the Compound 1-10 mg Group (administration of APAP, administration of 10 mg/kg Compound 1, open triangles); and the Positive Control Group (administration of APAP, administration of 150 mg/kg N-acetylcysteine “NAC”, open inverted triangles) * refers to P in the range from 0.01 to 0.05, ** refers to P in the range 0.001 to 0.01, and *** refers to P in the range from 0 to 0.001, based on one-way ANOVA; Tukey's multiple comparison test. All Compound 1 levels showed reduced ALT and AST biomarker levels after 24 hours APAP administration.

FIG. 6 depicts the effect of Compound 1 dosing on TNF-α, IL-1β, and IL-6 inflammation biomarker levels of the liver after 24 hrs administration of acetaminophen (APAP) as described in Example 1. Depicted from left to right on the x-axis are: the Control Group (no administration of APAP, black circles), the Vigilance Group (no administration of APAP, administration of 10 mg/kg Compound 1, black squares), the APAP Group (administration of APAP, black triangles), the Compound 1-1 mg Group (administration of APAP, administration of 1 mg/kg Compound 1, inverted black triangles); the Compound 1-2.5 mg Group (administration of APAP, administration of 2.5 mg/kg Compound 1, open circles); the Compound 1-5 mg Group (administration of APAP, administration of 5 mg/kg Compound 1, open squares); the Compound 1-10 mg Group (administration of APAP, administration of 10 mg/kg Compound 1, open triangles); and the Positive Control Group (administration of APAP, administration of 150 mg/kg N-acetylcysteine “NAC”, open inverted triangles) * refers to P in the range from 0.01 to 0.05, ** refers to P in the range 0.001 to 0.01, and *** refers to P in the range from 0 to 0.001, based on one-way ANOVA; Tukey's multiple comparison test. All Compound 1 levels showed reduced TNF-α, IL-1β, and/or IL-6 inflammation biomarker levels after 24 hours APAP administration.

FIG. 7 depicts the effect of Compound 1 on alcoholic liver disease (“ALD”) as described in Example 2. The features of ALD are scored according to the Non-alcoholic fatty liver disease (NAFLD) Activity Score (“NAS”) originally developed to measure changes in NAFLD during therapeutic trials but also applicable to alcoholic liver disease (“ALD”). Depicted from left to right on the x-axis are: the Control Group (no administration of alcohol, black squares), the ALD Group (administration of alcohol for 8 weeks plus binges, black triangles), and the Compound 1-w7 Group (administration of alcohol for 8 weeks plus binges, administration of 10 mg/kg Compound 1 at 7 weeks, inverted black triangles). Treatment with 10 mg/kg Compound 1 after 7 weeks reduces the ALD score compared to the untreated ALD Group.

FIG. 8 depicts the effect of Compound 1 on alcoholic liver disease (“ALD”) using Oil Red O staining as described in Example 2. Depicted on the y-axis is the Oil Red O (a.v.) quantification (a.v., “arbitrary value”, see Example 2). Depicted from left to right on the x-axis are: the Control Group of Pair Fed mice (no administration of alcohol, black circles), the ALD Group (administration of alcohol for 8 weeks plus binges, black squares), and the Compound 1-w7 Group (administration of alcohol for 8 weeks plus binges, administration of 10 mg/kg Compound 1 at 7 weeks, black triangles) *** refers to P in the range from 0 to 0.001, based on one-way ANOVA; Tukey's multiple comparison test. Treatment with 10 mg/kg Compound 1 after 7 weeks reduces hepatic lipid accumulation compared to the untreated ALD Group.

FIG. 9 depicts the effect of Compound 1 dosing on serum ALT (alanine aminotransferase) and AST (aspartate aminotransferase) biomarker levels of the liver in the alcoholic fatty liver disease (“ALD”) model as described in Example 2. Depicted from left to right on the x-axis are: the Control Group (no administration of alcohol, black circles), the ALD Group (administration of alcohol for 8 weeks plus binges, black squares), and the Compound 1-w7 Group (administration of alcohol for 8 weeks plus binges, administration of 10 mg/kg Compound 1 at 7 weeks, black triangles) *** refers to P in the range from 0 to 0.001, based on one-way ANOVA; Tukey's multiple comparison test. Treatment with 10 mg/kg Compound 1 after 7 weeks reduces ALT and AST levels compared to the untreated ALD.

### DETAILED DESCRIPTION OF THE INVENTION

In a first aspect, the present disclosure relates to Compound 1 or a pharmaceutically acceptable salt thereof

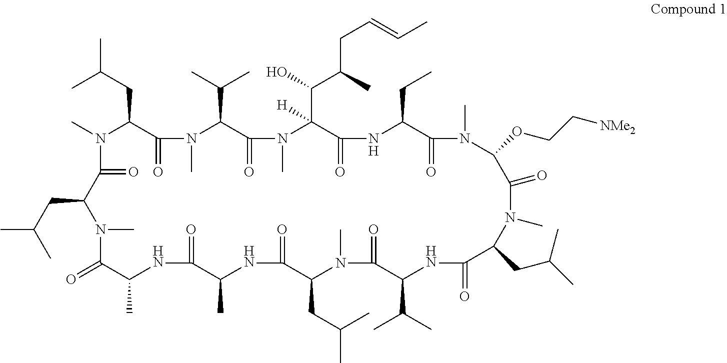 [SMILES: C/C=C/C[C@@H](C)[C@@H](O)[C@H]1C(=O)N[C@@H](CC)C(=O)N(C)[C@H](OCCN(C)C)C(=O)N(C)[C@@H](CC(C)C)C(=O)N[C@@H](C(C)C)C(=O)N(C)[C@@H](CC(C)C)C(=O)N[C@@H](C)C(=O)N[C@H](C)C(=O)N(C)[C@@H](CC(C)C)C(=O)N(C)[C@@H](CC(C)C)C(=O)N(C)[C@@H](C(C)C)C(=O)N1C]

for use in the prevention and/or treatment of a liver condition or disease in a subject exposed to an agent capable of inducing the liver condition or disease.

Compound 1 is a derivative of cyclosporin A, substituted at position 3 (sarcosine, otherwise known as N-methylglycine) by a N,N-dimethylaminoethoxy residue, and its synthesis and preparation has been described, for example in WO2019/0169572. Compound 1 as depicted above may be referred to in accordance with one of its chemical names as [(R)-2′(2-dimethylaminoethoxy)-Sar]³ cyclosporin A. It is to be understood herein that the Compound 1 as described within the context of the present disclosure, may refer also to in addition to any pharmaceutically acceptable salt thereof, also its enantiomer, diastereomers or racemates as well as its polymorphs, hydrates, or complexes. In an alternative and optional embodiment, Compound 1 may be provided as a mixture of the (R) and (S) stereoisomers at the 3-sarcosine position. Included is also the use of an optically pure stereoisomer of Compound 1, as well as the use of a combination of its stereoisomers. The phrase ‘optically pure’, interchangeable with the term ‘stereochemically pure’ refers to a compound having a level of stereochemical purity as recognized by one skilled in the art, based on conventional methods for the determination of stereochemistry and stereochemical purity. In a further and optional embodiment, Compound 1 or a pharmaceutically acceptable salt thereof may be provided as an isotope, for example where one or more of its atoms is replaced with an isotope such as ¹³C, or with deuterium.

As used herein, the term ‘agent’ includes compounds or substances or mixtures of compounds or substances capable of disrupting, impairing, or reducing liver function. Such compounds or substances include active pharmaceutical ingredients, and/or pharmacologically- or diagnostically-active compounds or mixtures of compounds useful for medical or therapeutic applications in the prevention, diagnosis, stabilization, treatment or management of a condition, disorder, or disease. An agent may be provided or administered to a subject as a medicament or pharmaceutical dosage form comprising one or more agents, and one or more non-pharmacologically active excipients or carriers. Other agents include small molecule drugs, biological drugs, traditional Chinese medicines, herbal remedies, natural medicines, and dietary supplements.

As understood herein, the phrase ‘exposure to an agent’ or similar, may refer to the exposure of a subject to an agent during the course of a treatment for a condition, symptom, or disease, wherein a subject is administered for therapeutic or diagnostic purposes, one or more doses of an agent such as any one or combination of the agents as defined in various embodiments herein. The phrase ‘exposure to an agent’ as used herein also comprises any unintended exposure of a subject to an agent, for example, but not limited to, accidental exposure such as from a needle-stick injury, or situational/unanticipated circumstances such as any physical or prolonged exposure to the agent. The phrase also includes intended exposures to agents, such as consumption of the agent, particularly in the context of the subject consuming a beverage such as an alcoholic beverage.

In one embodiment, the agent is an alcohol. The alcohol may be any organic compound containing one or more alcohol moieties that may cause a liver condition or disease upon exposure such as methanol, ethanol, propanol, isopropanol, butanol, isobutanol, and the like. Mixtures of alcohols are also contemplated. In a specific embodiment, the agent is a beverage alcohol, in particular, ethanol.

In another embodiment, the agent is a therapeutic agent. In specific embodiments, the therapeutic agent is selected from the group consisting of analgesics, antipyretics, anaesthetics, non-steroidal anti-inflammatories, antimicrobials, antibiotics, antifungals, antivirals, antiretrovirals, HIV therapies, hypoglycemics, lipid-lowering agents, herbal remedies, traditional Chinese medicine remedies, anticancer agents, antituberculosis agents, and cardiac medications. In a specific embodiment, the agent is the analgesic acetaminophen (APAP).

In some embodiments, the therapeutic agent is a traditional Chinese medicine remedy. Such traditional Chinese medicine remedies include herbal medicines or medicines derived from animals, minerals, or other substances. In some embodiments, the traditional Chinese medicine remedy is selected from the group consisting of Polygonum multiflorum Thunb. (Heshouwu in Chinese) Tripterygium wilfordii Hook. f. (Leigongteng in Chinese), Bupleurum chinense DC. (Chaihu in Chinese), Epimedium brevicornum Maxim. (Yinyanghuo in Chinese), Silybum marianum (L.) Gaertn. (Shuifeiji in Chinese), Cassia acutifolia Delile (Fanxieye in Chinese), Rhamnus purshiana DC. (cascara) (Shuli in Chinese), Anisi Stellati Fructus (Bajiao in Chinese), Radix sanguisorbae (Diyu in Chinese), Fructus Gardeniae (Zhizi in Chinese), Galla Chinensis (Wubeizi in Chinese), Cortex Granati (Shiliupi in Chinese), Chebulae Fructus (Hezi in Chinese), Rhizoma Acori tatarinowii (Shichangpu in Chinese), Fructus Foeniculi (Xiaohuixiang in Chinese), Cortex Cinnamomi (Guipi in Chinese), Fructus Aristolochiae (Madouling in Chinese), Caulis Akebiae (Mutong in Chinese), Cortex Albiziae (Hehuanpi in Chinese), Semen Strychni (Maqianzi in Chinese), Rhizoma Alismatis (Zexie in Chinese), Folium Sennae (Fanxieye in Chinese), Semen Cassiae (Juemingzi in Chinese), Herba Chenopodii (Tujinjie in Chinese), Folium Clerodendri (Chouwutong in Chinese), Radix Dioscoreae bulbifera (Huangdu in Chinese), Omoto Nipponlily (Wannianqing in Chinese), Herba Ephedrae (Mahuang in Chinese), Herba Gynurae (Jusanqi in Chinese), Semen Hydnocarpi Hainanensis (Dafengzi in Chinese), Folium Ilexi pubescensi (Maodongqing in Chinese), Fructus Trichosanthis (Gualou in Chinese), Fructus Meliaceae (Kulianzi in Chinese), Semen Myristicae (Roudoukou in Chinese), Herba Papaveri somniferi (Yingsu in Chinese), Radix Phytolaccae (Shanglu in Chinese), Radix Polygoni cuspidati (Huzhang in Chinese), Radix Polygoni multiflori (Heshouwu in Chinese), Rhubarb (Dahuang in Chinese), Semen Ricini (Bimazi in Chinese), Radix et Rhizoma Salviae miltiorrhizae (Danshen in Chinese), Radix Scutellariae (Huangqin in Chinese), Herba Senecioe scandensi (Qianniguang in Chinese), Herba Scutellariae Barbatae (Banzhilian in Chinese), Radix Stephaniae tetrandrae (Hanfangji in Chinese), Flos Syzygii aromatici (Dingxiang in Chinese), Herba Taxilli (Sangjisheng in Chinese), Radix et Rhizoma Tripterygii (Leigongteng in Chinese), Herba Typhae (Xiangpu in Chinese), Radix Valerian (Xiecao in Chinese), Fructus Xanthii (Cangerzi in Chinese), Zhuangguguanjie pill, Baishi pill, Liushen pill, Shiduqing pill, Xuedu pill, Zhifeng-tougu pill, Xiaokechuan pill, Tianma pill, Xiaochaihu decoction, Dachaihu decoction, Gegen decoction, Dahuangmudanpi decoction, Complex Qingdai capsule, Baidianfeng capsule, Zhuangushenjin capsule, Diaoxinxuekang capsule, Xiaoying tablets, Xiaohe tablets, Guxian tablets, Zengshengping tablets, Niuhuang Jiedu tablets, Kunming-Shanhaitang tablets, Baifukang granule, Complex Danshen injection, Ganji powder, Fangfeng-Tongsheng powder, Radix Aconiti Kusnezoffii, Radix Aconiti penduli, Rhizoma Arisaematis, Flos Daturae, Rhizoma Typhonium, Radix Aconiti Lateralis, Rhizoma Pinellia, Semen Nut-vomitive, Herba Euphorbia, Radix euphorbiae lantu, Garcinia, Leptochloa, Radix Rhododendroni molli, Semen Hyoscyami, Croton, Venenum Bufonis, Cantharidin, Huechys sanguinea, Arsenic, Arsenolite, Arsenic stone, Rabiagar, Calomelas, Hydrargyri oxydum Rubrum, Mercury, Aconitine, febrifugine, ajmaline, vincristine, Cardiac glycosides, cyanogenic glycosides, saponins, bulbifera, toxin fruit, croton, castor, abrinjatropha, trichosanthes, centipede, snake venom, viper, Toosendan, spurge, Coriaria leaf, wormwood leaves, Gall, peel, holly leaf, Cinnabar, realgar, light powder, litharge, vitriol, red lead, Garcinia, red fennel root, Hydnocarpus, Pinellia ternata, daphne, melon pedicle acids, Asarum, rue, mint, Asarum forbesii, musk, grass, and any mixture or combination thereof.

Compound 1 or a pharmaceutically acceptable salt thereof may be used for the prevention and/or treatment of a liver condition or disease in a subject exposed to an agent capable of inducing the liver condition or disease. In some embodiments, the liver condition or disease to be prevented and/or treated is a hepatotoxic condition or disease. The liver condition or disease may be the result of direct toxicity of the agent, immune-mediated mechanisms, or a combination of direct toxicity of the agent and immune-mediated mechanisms. In some embodiments, the immune-mediated mechanism is an inflammatory reaction. In particular embodiments, the hepatotoxic condition or disease is selected from the group consisting of toxic hepatitis, toxin-induced hepatitis, drug-induced hepatitis, toxin-induced hepatic necrosis, drug-induced hepatic necrosis, toxin-induced hepatic fibrosis, drug-induced hepatic fibrosis, toxin-induced hepatic granuloma, drug-induced hepatic granuloma, toxic liver disease, toxin-induced liver disease, drug-induced liver disease, toxic liver disease with hepatitis, toxin-induced liver disease with hepatitis, drug-induced liver disease with hepatitis, toxin-induced liver damage, drug-induced liver damage, hepatogenous poisoning, toxic liver disease with cholestasis, or a combination thereof. In some embodiments, the liver condition or disease is an acute or chronic liver condition or disease. In particular embodiments, the liver condition or disease is an acute condition or disease. In other particular embodiments, the liver condition or disease is a chronic condition or disease.

In a particular embodiment, the liver condition or disease is a liver condition or disease arising from exposure to acetaminophen (also known as N-acetyl-p-aminophenol, APAP, paracetamol, and Tylenol®). Exposure to acetaminophen may cause dose-related hepatocellular necrosis and/or acute liver failure. An acetaminophen overdose may be intentional or unintentional. The toxicity is characterized as “acute” or “hyperacute” resulting in abdominal pain and nausea after about 12 to 24 hours after overdose. Levels of aminotransferases such as aspartate aminotransferase (AST) and alanine aminotransferase (ALT) increase rapidly to elevated levels. As the overdose progresses, the subject may experience other severe symptoms such as hyperammonemia, somnolence, stupor, coma, lactic acidosis, cerebral oedema, brain stem herniation, and vascular collapse. Other organs, such as the kidney, may fail in patients with acute liver failure. Central lobular hepatocellular necrosis is a distinct indicator of acetaminophen injury. Acetaminophen overdoses are commonly treated with N-acetylcysteine (NAC).

In another particular embodiment, the liver condition or disease is a liver condition or disease arising from exposure to alcohol. Alcoholic liver disease generally progresses from alcoholic fatty liver (AFL, also known as steatosis) to alcoholic steatohepatitis (ASH) which is characterized by hepatic inflammation. Chronic ASH may eventually lead to more serious liver conditions including fibrosis, cirrhosis, necrosis, in particular focal necrosis, and in some cases hepatocellular cancer (HCC). Moreover, severe ASH (with or without cirrhosis) can lead to alcoholic hepatitis, which is an acute representation of ALD that is associated with liver failure and high incidence of death. In some embodiments, the liver condition or disease associated with exposure to alcohol is non-alcoholic fatty liver disease, alcoholic liver disease, alcoholic fatty liver disease (steatosis), alcoholic steatohepatitis, hepatic inflammation, alcoholic hepatitis, fibrosis, cirrhosis, hepatocellular cancer, or any combination thereof. In some embodiments, administration of Compound 1 to the subject treats or prevents the alcohol-induced condition or disease. In a particular embodiment, Compound 1 reduces a non-alcoholic fatty liver disease activity score (NAS) of the subject relative to an untreated subject. As the features of alcoholic liver disease (“ALD”) can be scored using the non-alcoholic fatty liver disease (NAFLD) Activity Score (“NAS”) method, which was originally developed to measure changes in NAFLD, NAS scoring can be used to determine the treatment or prophylactic efficacy of Compound 1 in relation to ALD. In another particular embodiment, Compound 1 effectively alleviates the damage to the liver due to excessive alcohol ingestion, as revealed by a reduction of Oil Red O liver staining of the subject relative to an untreated subject.

In some embodiments, the subject has a pre-existing condition or disease that increases the subject's risk of developing a liver condition or disease when exposed to the agent.

In some embodiments, the agent induces oxidative stress in the liver. In some embodiments, Compound 1 reduces the oxidative stress of the liver of the subject relative to an untreated subject. In a particular embodiment, Compound 1 reduces the presence of an oxidative stress biomarker selected from the group consisting of CAT, SOD, NRF2, iNOS, or any combination thereof relative to an untreated subject.

In some embodiments, the agent induces necrosis of the liver. In some embodiments, Compound 1 reduces the necrosis of the liver of the subject relative to an untreated subject. In a particular embodiment, Compound 1 reduces the presence of a necrosis biomarker selected from the group consisting of BCL-2, BAX, or a combination thereof relative to an untreated subject. In another particular embodiment, the necrosis of the liver is a central lobular necrosis of the liver. In yet another particular embodiment, the necrosis of the liver is a focal necrosis of the liver.

In some embodiments, the agent induces inflammation of the liver. In some embodiments, Compound 1 reduces the inflammation of the liver relative to an untreated subject. In a particular embodiment, Compound 1 reduces the presence of an inflammation biomarker selected from the group consisting of p38 MAPK, NF-κB p65, TNF-α, IL-1β, IL-6, and IL-10. In another particular embodiment, Compound 1 reduces the presence an inflammation biomarker selected from the group consisting of TNF-α, IL-1β, and IL-6.

In some embodiments, the agent induces an elevation of ALT (alanine aminotransferase) and AST (aspartate aminotransferase) biomarkers. In some embodiments, Compound 1 reduces the elevation of ALT and AST biomarkers relative to an untreated subject.

As used herein, the term ‘treating’ or ‘treatment’ which may be used interchangeably with the term ‘therapy’ relates to a therapeutic intervention capable of effecting a cure, improvement, amelioration, control, control of progression, prevention of progression of a disease or a condition or symptom associated with said disease or condition.

As understood herein the term ‘prevention’, which may be used interchangeably with the term ‘prophylaxis’ refers to the use of a compound, or composition, for preventing the occurrence of a disease, condition or symptom, or significantly reducing the likelihood of occurrence of a disease, condition or symptom, as well as the prevention of, for example, a further reoccurrence of a disease, condition or associated symptom. Also included within the meaning of the term is the prevention of progression of a disease, condition, or associated symptom, after an initial improvement or after initial removal of the cause of the disease, condition, or symptom.

It may also be understood that any one, or combination of uses, and methods of treatment or prophylaxis as described herein comprises the administration to a subject in need thereof, a pharmacologically effective amount of Compound 1. A pharmacologically effective amount refers to an amount, dose, concentration or strength of the compound or compound equivalent which is useful or capable of producing or contributing to a desired therapeutically and/or prophylactically-relevant pharmacological effect. The term ‘pharmacologically effective amount’ may also be used interchangeably with the term ‘therapeutically effective amount’.

As understood herein, the term ‘subject’ may be used interchangeably with the term ‘patient’. In one preferred embodiment, the subject is a human subject. In an optional embodiment, the subject may be other animals, such as other mammals. In an optional embodiment, the invention may also have application, for instance, in farm animals or other veterinary subjects, in particular mammals such cats, dogs, primates, horses, cows, and pigs.

Compound 1, or a pharmaceutically acceptable salt thereof may be administered, in one embodiment, to a subject repeatedly, e.g., more than once during its prescribed course of treatment. Doses of Compound 1 may thus be administered in a period between successive doses of the agent. Optionally, more than one dose of Compound 1 may be administered in a period between successive doses of an agent.

As used herein, the term ‘dose’ or ‘dosage’ as such refers to a single, or unit dose of Compound 1 or a pharmaceutically acceptable salt thereof, or agent or drug substance, unless prefaced or followed by an indication of time, time interval or indication of quantity. A ‘daily dose’ or ‘dosage per day’ for example refers to the total dose amount of Compound 1 or agent or drug substance administered in the course of one day (24 hours). A daily dose may comprise only one dose, if only one dose is administered once per day but may also be a total based on the sum of multiple unit doses that administered during a day, for example, if more than one unit dose is administered at two or more timed intervals during a day. Intervals between doses may be, for example, two doses administered approximately every 12 hours, or three doses administered approximately every 8 hours. Also as used herein a dose of Compound 1 refers to a unit dose of Compound 1, or a pharmaceutically acceptable salt thereof, but may also be applicable to a medicament, or composition or dosage form comprising said unit dose of Compound 1, or a pharmaceutically acceptable salt thereof.

In another embodiment, Compound 1 or a pharmaceutically acceptable salt thereof is administered to a subject, preferably a human subject, at a dose of about 0.001 to 5 mg/kg, or at a dose of about 0.001 to 10 mg/kg. Optionally, Compound 1 may be administered to a subject, preferably a human subject at a dose of about 0.1 to 5 mg/kg, or 0.1 to 10 mg/kg. Said dose amount refers to a single dose. A single dose may be administered more than once during a course of treatment. Dose amounts, if described in respect of any pharmaceutically acceptable salts of Compound 1 herein refer to Compound 1 equivalents. A single dose of Compound 1 moreover, may be administered to a subject by administering a composition or medicament provided in a pharmaceutically acceptable dosage form, and comprising a dose of Compound 1.

In some embodiments, Compound 1 or a pharmaceutically acceptable salt thereof is administered to a subject, preferably a human subject, within hours, in some cases within one hour after exposure to the agent. In other embodiments, Compound 1 or a pharmaceutically acceptable salt thereof is administered to a subject, preferably a human subject, within weeks after exposure of the agent, in some cases six to ten weeks after exposure, especially when the agent is alcohol, and the subject has been chronically consuming alcohol.

When used herein the term ‘about’ or the like in connection with an attribute or value such as dose amount includes the exact attribute or precise value, as well as any attribute or value typically considered to fall within the normal or accepted variability associated with the technical field, and methods of measuring or determining said attribute or value. The term allows for any variation which in the common practice would allow for the product being evaluated to be considered bioequivalent in a mammal to the recited strength or dose of a claimed product.

The use of Compound 1 or a pharmaceutically acceptable salt thereof and method of treatment or prevention as described in any one of the embodiments herein is also provided in the context of the present disclosure, for the manufacture or preparation of a medicament or medicine adapted and prescribed for said uses or methods of treatment or prophylaxis.

A pharmaceutically acceptable salt of Compound 1 is a salt which retains its biological properties and which is non-toxic and is compatible for pharmaceutical use. Salts of the invention may result from the addition of acids to the Compound 1. The resultant acid addition salts include those formed with acetic, 2,2-dichloroacetic, citric, lactic, mandelic, glycolic, adipic, alginic, aryl sulfonic acids (e.g., benzenesulfonic, naphthalene-2-sulfonic, naphthalene-1,5-disulfonic and p-toluenesulfonic), ascorbic (e.g. L-ascorbic), L-aspartic, benzoic, 4-acetamidobenzoic, butanoic, (+)-camphoric, camphor-sulfonic, (+)-(1S)-camphor-10-sulfonic, capric, caproic, caprylic, cinnamic, citric, cyclamic, dodecylsulfuric, ethane-1,2-disulfonic, ethanesulfonic, 2-hydroxyethanesulfonic, formic, fumaric, galactaric, gentisic, glucoheptonic, gluconic (e.g. D-gluconic), glucuronic (e.g. D-glucuronic), glutamic (e.g. L-glutamic), α-oxoglutaric, glycolic, hippuric, hydrobromic, hydrochloric, hydriodic, isethionic, lactic (e.g. (+)-L-lactic and (±)-DL-lactic), lactobionic, maleic, malic (e.g. (−)-L-malic), (±)-DL-mandelic, metaphosphoric, methanesulfonic, 1-hydroxy-2-naphthoic, nicotinic, nitric, oleic, orotic, oxalic, palmitic, pamoic, phosphoric, propionic, L-pyroglutamic, salicylic, 4-amino-salicylic, sebacic, stearic, succinic, sulfuric, tannic, tartaric (e.g. (+)-L-tartaric), thiocyanic, undecylenic and valeric acids. In particular acid addition salts include those derived from mineral acids such as hydrochloric, hydrobromic, phosphoric, metaphosphoric, nitric and sulfuric acids; from organic acids, such as tartaric, acetic, citric, malic, lactic, fumaric, benzoic, glycolic, gluconic, succinic and arylsulfonic acids.

Compound 1 or a pharmaceutically acceptable salt thereof may be administered enterally or parenterally to a subject. In one embodiment, Compound 1, or a composition or a medicament comprising said Compound 1 is adapted for administration or is administered parenterally, for example by intravenous injection or by subcutaneous, or intramuscular injection, or by intravenous or subcutaneous infusion. In an alternative embodiment, Compound 1, or a composition or medicament comprising Compound 1 is adapted for administration, or is administered to a subject enterally, for example orally.

The present invention may also relate to a medicament, or a pharmaceutical composition comprising a Compound 1 or a pharmaceutically acceptable salt thereof according to any one or combination of the embodiments described herein above, and one or more pharmaceutically acceptable excipients. The medicament, or pharmaceutical composition comprising said Compound 1 may be formulated in a dosage form suitable or adapted for injection or infusion by any of the administration methods above. Alternatively, for oral administration, the medicament or pharmaceutical composition comprising Compound 1 may be provided in a dosage form suitable or adapted for oral administration, for example such as, but not limited to a tablet, capsule, gel cap, or film. Said medicament, or pharmaceutical composition may be used in accordance with any of the methods of treatment or prevention or uses described herein.

In one embodiment, wherein Compound 1 is formulated in a dosage form suitable or adapted for injection or infusion, the formulation may be provided in the form of a clear solution, or as a lyophilized or freeze-dried powder, from which a clear solution may be obtainable, by reconstitution in a solvent or solution suitable, i.e., formulated for reconstitution. In one embodiment, said reconstitution solvent may comprise a solubilizer and/or a surfactant. In one specific embodiment, the reconstitution solution may comprise a polysorbate, e.g., polysorbate 80 or Tween 80; further optionally said solvent has a pH of between 3.5 and 4.5, or a pH between 4.2 and 4.5. In another embodiment, a medicament, or a pharmaceutical composition comprising Compound 1 or a pharmaceutically acceptable salt thereof may further comprise a polysorbate. The composition, in one embodiment, is a clear aqueous solution, with a pH between about 3.5 and about 4.5. In another embodiment, the solution has a pH between about 4.2 and about 4.5. In yet another embodiment, the composition comprising Compound 1 or a salt thereof further comprises a polysorbate and optionally one or more further excipients, for example wherein the excipient is an acid (e.g., an organic acid) and/or one or more buffering agents. The polysorbate (e.g., a polyoxyethylene sorbitan monoester) is in one embodiment, polysorbate 80, or Tween 80 (polyoxyethylene sorbitan monooleate). In one embodiment, the composition comprises, or consists of an effective amount of Compound 1 or a salt thereof: a polysorbate, acetic acid, and an acetate salt, e.g., sodium acetate.

In another embodiment, Compound 1 is formulated with a suitable acid. For example, Compound 1 may be formulated with an acid selected from succinic acid, L-malic acid, or phosphoric acid. In yet a further embodiment, a composition comprising Compound 1 may further comprise a solubilizer and/or a surfactant, such as a polyethylene glycol, e.g., PEG 400, or a polysorbate e.g., Tween 80. In one specific embodiment, a composition comprising Compound 1 may further comprise of an acid e.g., succinic acid, L-malic acid, phosphoric acid or acetic acid, and a surfactant and/or a solubilizer, such as polyethylene glycol, e.g., PEG 400, or a polysorbate, e.g., Tween 80. The composition may, in one embodiment, be suitable or adapted for bolus injection, and may be provided in the form of a clear aqueous solution with pH between about 5.4 and about 5.6.

Compositions which are suitable for injection or infusion are generally in the form of solutions, meaning that formulation components, including active ingredients, must be fully dissolved or solubilized. The formation or presence of micellar phases or any other type of phase separation of any one of the components or active ingredients is not preferred, either at the point of administration, or optionally, if the composition is stored as a solution, during storage. In one embodiment, a composition according to the present disclosure as described herein and comprising Compound 1 or a salt thereof, is in the form of a clear solution. Preferably, the composition is in the form of a stable and clear solution, e.g., stable for at least 1 hour, or at least 15 hours at room temperature (or in cold storage such as at 0° C.). The stability of a solution may, for example, be determined by visual inspection (e.g., for phase separation or other visible changes), or additionally, based on turbidity detector measurements.

Optionally, the medicament, or pharmaceutical composition comprising Compound 1 or a pharmaceutically acceptable salt thereof may also further comprise a drug substance, i.e., Compound 1 may be co-formulated as a single composition or dosage form together with another drug substance.

The present invention may in a further aspect, also relate to a kit comprising a composition comprising Compound 1 or a pharmaceutically acceptable salt thereof and optionally instructions for the administration of Compound 1 or a salt thereof as described in any one of the methods or uses described herein.

The following list of numbered items are embodiments comprised in the present disclosure:
1. Use of Compound 1 or a pharmaceutically acceptable salt thereof

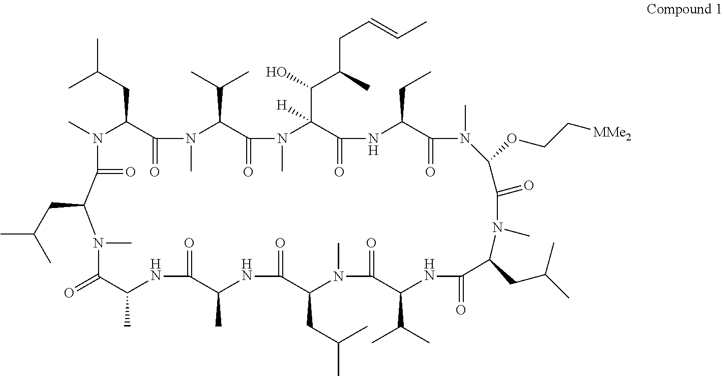

in the manufacture of a medicament for the prevention and/or treatment of a liver condition or disease in a subject exposed to an agent capable of inducing the liver condition or disease.
2. The use according to item 1, wherein the liver condition or disease is a hepatotoxic condition or disease.
3. The use according to item 2, wherein the hepatotoxic condition or disease is selected from the group consisting of toxic hepatitis, toxin-induced hepatitis, drug-induced hepatitis, toxin-induced hepatic necrosis, drug-induced hepatic necrosis, toxin-induced hepatic fibrosis, drug-induced hepatic fibrosis, toxin-induced hepatic granuloma, drug-induced hepatic granuloma, toxic liver disease, toxin-induced liver disease, drug-induced liver disease, toxic liver disease with hepatitis, toxin-induced liver disease with hepatitis, drug-induced liver disease with hepatitis, toxin-induced liver damage, drug-induced liver damage, hepatogenous poisoning, toxic liver disease with cholestasis, or a combination thereof.
4. The use according to any one of items 1 to 3, wherein the liver condition or disease is an acute or chronic liver condition or disease.
5. The use according to any one of items 1 to 4, wherein the liver condition or disease is an acute liver condition or disease.
6. The use according to any one of items 1 to 4, wherein the liver condition or disease is a chronic liver condition or disease.
7. The use according to any one of items 1 to 6, wherein the liver condition or disease is the result of direct toxicity of the agent, immune-mediated mechanisms, or a combination of direct toxicity of the agent and immune-mediated mechanisms.
8. The use according to item 7, wherein the immune-mediated mechanism is an inflammatory reaction.
9. The use according to any one of items 1 to 8, wherein the agent is an alcohol.
10. The use according to item 9, wherein the alcohol is a beverage alcohol.
11. The use according to any one of items 9 to 10, wherein the alcohol is ethanol.
12. The use according to any one of items 9 to 11, wherein Compound 1 reduces a non-alcoholic fatty liver disease activity score of the subject relative to an untreated subject.
13. The use according to any one of items 9 to 12, wherein Compound 1 reduces Oil Red O liver staining of the subject relative to an untreated subject.
14. The use according to any one of items 9 to 13, wherein the liver condition or disease is non-alcoholic fatty liver disease, alcoholic liver disease, alcoholic fatty liver disease (steatosis), alcoholic steatohepatitis, hepatic inflammation, alcoholic hepatitis, fibrosis, cirrhosis, hepatocellular cancer, or any combination thereof.
15. The use according to any one of items 1 to 8, wherein the agent is a therapeutic agent.
16. The use according to item 15, wherein the therapeutic agent is selected from the group consisting of analgesics, antipyretics, anesthetics, non-steroidal anti-inflammatories, antimicrobials, antibiotics, antifungals, antivirals, antiretrovirals, HIV therapies, hypoglycemics, lipid-lowering agents, herbal remedies, traditional Chinese medicine remedies, anticancer agents, antituberculosis agents, and cardiac medications.
17. The use according to item 15 or 16, wherein the therapeutic agent is acetaminophen.
18. The use according to any one of the preceding items, wherein the subject has a pre-existing condition or disease that increases the subject's risk of developing a liver condition or disease when exposed to the agent.
19. The use according to any one of the preceding items, wherein the agent induces oxidative stress in the liver.
20. The use according to item 19, wherein Compound 1 reduces the oxidative stress of the liver of the subject relative to an untreated subject.
21. The use according to item 20, wherein Compound 1 reduces the presence of an oxidative stress biomarker selected from the group consisting of CAT, SOD, NRF2, iNOS, or any combination thereof relative to an untreated subject.
22. The use according to any one of the preceding items, wherein the agent induces necrosis of the liver.
23. The use according to item 22, wherein Compound 1 reduces the necrosis of the liver of the subject relative to an untreated subject.
24. The use according to item 23, wherein Compound 1 reduces the presence of a necrosis biomarker selected from the group consisting of BCL-2, BAX, or a combination thereof relative to an untreated subject.
25. The use according to any one of items 22 to 24, wherein the necrosis of the liver is a central lobular necrosis of the liver.
26. The use according to any one of items 22 to 25, wherein the necrosis of the liver is a focal necrosis of the liver.
27. The use according to any one of the preceding items, wherein the agent induces inflammation of the liver.
28. The use according to item 27, wherein Compound 1 reduces the inflammation of the liver of the subject relative to an untreated subject.
29. The use according to item 28, wherein Compound 1 reduces the presence of an inflammation biomarker selected from the group consisting of p38 MAPK, NF-κB p65, TNF-α, IL-1β, IL-6, and IL-10.
30. The use according to item 29, wherein Compound 1 reduces the presence of an inflammation biomarker selected from the group consisting of TNF-α, IL-18, and IL-6.
31. The use according to any one of the preceding items, wherein the agent induces an elevation of ALT and AST biomarkers.
32. The use according to item 31, wherein Compound 1 reduces the elevation of ALT and AST biomarkers relative to an untreated subject.
33. The use according to any one of the preceding items, wherein Compound 1 is administered at a dosage of about 0.001 to about 10 mg/kg, about 0.1 to about 10 mg/kg, or about 5 mg/kg to about 10 mg/kg.
34. The use according to any one of the preceding items, wherein the subject is a human subject.
35. The use according to any one of the preceding items, wherein the medicament is formulated for administration by injection or infusion, preferably subcutaneous, intramuscular, or intravenous injection, or intravenous or subcutaneous infusion.
36. The use according to any of the preceding items, wherein the medicament is formulated for administration by injection or infusion, preferably intravenous injection or infusion.
37. Compound 1 or a pharmaceutically acceptable salt thereof

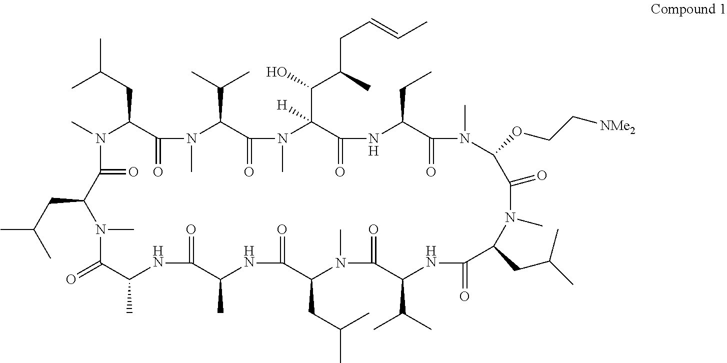 [SMILES: C/C=C/C[C@@H](C)[C@@H](O)[C@H]1C(=O)N[C@@H](CC)C(=O)N(C)[C@H](OCCN(C)C)C(=O)N(C)[C@@H](CC(C)C)C(=O)N[C@@H](C(C)C)C(=O)N(C)[C@@H](CC(C)C)C(=O)N[C@@H](C)C(=O)N[C@H](C)C(=O)N(C)[C@@H](CC(C)C)C(=O)N(C)[C@@H](CC(C)C)C(=O)N(C)[C@@H](C(C)C)C(=O)N1C]

for use in the prevention and/or treatment of a liver condition or disease in a subject exposed to an agent capable of inducing the liver condition or disease.
38. The compound for use according to item 37, wherein the liver condition or disease is a hepatotoxic condition or disease.
39. The compound for use according to item 38, wherein the hepatotoxic condition or disease is selected from the group consisting of toxic hepatitis, toxin-induced hepatitis, drug-induced hepatitis, toxin-induced hepatic necrosis, drug-induced hepatic necrosis, toxin-induced hepatic fibrosis, drug-induced hepatic fibrosis, toxin-induced hepatic granuloma, drug-induced hepatic granuloma, toxic liver disease, toxin-induced liver disease, drug-induced liver disease, toxic liver disease with hepatitis, toxin-induced liver disease with hepatitis, drug-induced liver disease with hepatitis, toxin-induced liver damage, drug-induced liver damage, hepatogenous poisoning, toxic liver disease with cholestasis, or a combination thereof.
40. The compound for use according to any one of items 37 to 39, wherein the liver condition or disease is an acute or chronic liver condition or disease.
41. The compound for use according to any one of items 37 to 40, wherein the liver condition or disease is an acute liver condition or disease.
42. The compound for use according to any one of items 37 to 40, wherein the liver condition or disease is a chronic liver condition or disease.
43. The compound for use according to any one of items 37 to 42, wherein the liver condition or disease is the result of direct toxicity of the agent, immune-mediated mechanisms, or a combination of direct toxicity of the agent and immune-mediated mechanisms.
44. The compound for use according to item 43, wherein the immune-mediated mechanism is an inflammatory reaction.
45. The compound for use according to any one of items 37 to 44, wherein the agent is an alcohol.
46. The compound for use according to item 45, wherein the alcohol is a beverage alcohol.
47. The compound for use according to any one of items 45 to 46, wherein the alcohol is ethanol.
48. The compound for use according to any one of items 45 to 47, wherein Compound 1 reduces a non-alcoholic fatty liver disease activity score of the subject relative to an untreated subject.
49. The compound for use according to any one of items 45 to 48, wherein Compound 1 reduces Oil Red O liver staining of the subject relative to an untreated subject.
50. The compound for use according to any one of items 45 to 49, wherein the liver condition or disease is non-alcoholic fatty liver disease, alcoholic liver disease, alcoholic fatty liver disease (steatosis), alcoholic steatohepatitis, hepatic inflammation, alcoholic hepatitis, fibrosis, cirrhosis, hepatocellular cancer, or any combination thereof.
51. The compound for use according to any one of items 37 to 44, wherein the agent is a therapeutic agent.
52. The compound for use according to item 51, wherein the therapeutic agent is selected from the group consisting of analgesics, antipyretics, anesthetics, non-steroidal anti-inflammatoires, antimicrobials, antibiotics, antifungals, antivirals, antiretrovirals, HIV therapies, hypoglycemics, lipid-lowering agents, herbal remedies, traditional Chinese medicine remedies, anticancer agents, antituberculosis agents, and cardiac medications.
53. The compound for use according to item 51 or 52, wherein the therapeutic agent is acetaminophen.
54. The compound for use according to any one of the preceding items, wherein the subject has a pre-existing condition or disease that increases the subject's risk of developing a liver condition or disease when exposed to the agent.
55. The compound for use according to any one of the preceding items, wherein the agent induces oxidative stress in the liver.
56. The compound for use according to item 55, wherein Compound 1 reduces the oxidative stress of the liver of the subject relative to an untreated subject.
57. The compound for use according to item 56, wherein Compound 1 reduces the presence of an oxidative stress biomarker selected from the group consisting of CAT, SOD, NRF2, iNOS, or any combination thereof relative to an untreated subject.
58. The compound for use according to any one of the preceding items, wherein the agent induces necrosis of the liver.
59. The compound for use according to item 58, wherein Compound 1 reduces the necrosis of the liver of the subject relative to an untreated subject.
60. The compound for use according to item 59, wherein Compound 1 reduces the presence of a necrosis biomarker selected from the group consisting of BCL-2, BAX, or a combination thereof relative to an untreated subject.
61. The compound for use according to any one of items 58 to 60, wherein the necrosis of the liver is a central lobular necrosis of the liver.
62. The compound for use according to any one of items 58 to 61, wherein the necrosis of the liver is a focal necrosis of the liver.
63. The compound for use according to any one of the preceding items, wherein the agent induces inflammation of the liver.
64. The compound for use according to item 63, wherein Compound 1 reduces the inflammation of the liver of the subject relative to an untreated subject.
65. The compound for use according to item 64, wherein Compound 1 reduces the presence of an inflammation biomarker selected from the group consisting of p38 MAPK, NF-κB p65, TNF-α, IL-1β, IL-6, and IL-10.
66. The compound for use according to item 65, wherein Compound 1 reduces the presence of an inflammation biomarker selected from the group consisting of TNF-α, IL-1β, and IL-6.
67. The compound for use according to any one of the preceding items, wherein the agent induces an elevation of ALT and AST biomarkers.
68. The compound for use according to item 67, wherein Compound 1 reduces the elevation of ALT and AST biomarkers relative to an untreated subject.
69. The compound for use according to any one of the preceding items, wherein Compound 1 is administered at a dosage of about 0.001 to about 10 mg/kg, about 0.1 to about 10 mg/kg, or about 5 mg/kg to about 10 mg/kg.
70. The compound for use according to any one of the preceding items, wherein the subject is a human subject.
71. The compound for use according to any one of the preceding items, wherein the compound is formulated for administration by injection or infusion, preferably subcutaneous, intramuscular, or intravenous injection, or intravenous or subcutaneous infusion.
72. The compound for use according to any of the preceding items, wherein the compound is formulated for administration by injection or infusion, preferably intravenous injection or infusion.
73. A method for preventing and/or treating a liver condition or disease in a subject exposed to an agent capable of inducing the liver condition or disease, the method comprising administering to said subject Compound 1 or a pharmaceutically acceptable salt thereof

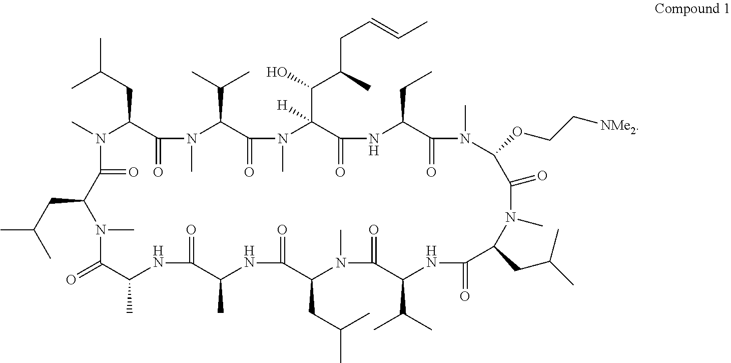 [SMILES: C/C=C/C[C@@H](C)[C@@H](O)[C@H]1C(=O)N[C@@H](CC)C(=O)N(C)[C@H](OCCN(C)C)C(=O)N(C)[C@@H](CC(C)C)C(=O)N[C@@H](C(C)C)C(=O)N(C)[C@@H](CC(C)C)C(=O)N[C@@H](C)C(=O)N[C@H](C)C(=O)N(C)[C@@H](CC(C)C)C(=O)N(C)[C@@H](CC(C)C)C(=O)N(C)[C@@H](C(C)C)C(=O)N1C]

74. The method according to item 73, wherein the liver condition or disease is a hepatotoxic condition or disease.
75. The method according to item 74, wherein the hepatotoxic condition or disease is selected from the group consisting of toxic hepatitis, toxin-induced hepatitis, drug-induced hepatitis, toxin-induced hepatic necrosis, drug-induced hepatic necrosis, toxin-induced hepatic fibrosis, drug-induced hepatic fibrosis, toxin-induced hepatic granuloma, drug-induced hepatic granuloma, toxic liver disease, toxin-induced liver disease, drug-induced liver disease, toxic liver disease with hepatitis, toxin-induced liver disease with hepatitis, drug-induced liver disease with hepatitis, toxin-induced liver damage, drug-induced liver damage, hepatogenous poisoning, toxic liver disease with cholestasis, or a combination thereof.
76. The method according to any one of items 73 to 75, wherein the liver condition or disease is an acute or chronic liver condition or disease.
77. The method according to any one of items 73 to 76, wherein the liver condition or disease is an acute liver condition or disease.
78. The method according to any one of items 73 to 76, wherein the liver condition or disease is a chronic liver condition or disease.
79. The method according to any one of items 73 to 78, wherein the liver condition or disease is the result of direct toxicity of the agent, immune-mediated mechanisms, or a combination of direct toxicity of the agent and immune-mediated mechanisms.
80. The method according to item 79, wherein the immune-mediated mechanism is an inflammatory reaction.
81. The method according to any one of items 73 to 80, wherein the agent is an alcohol.
82. The method according to item 81, wherein the alcohol is a beverage alcohol.
83. The method according to any one of items 81 to 82, wherein the alcohol is ethanol.
84. The method according to any one of items 81 to 83, wherein Compound 1 reduces a non-alcoholic fatty liver disease activity score of the subject relative to an untreated subject.
85. The method according to any one of items 81 to 84, wherein Compound 1 reduces Oil Red O liver staining of the subject relative to an untreated subject.
86. The method according to any one of items 81 to 85, wherein the liver condition or disease is non-alcoholic fatty liver disease, alcoholic liver disease, alcoholic fatty liver disease (steatosis), alcoholic steatohepatitis, hepatic inflammation, alcoholic hepatitis, fibrosis, cirrhosis, hepatocellular cancer, or any combination thereof.
87. The method according to any one of items 73 to 80, wherein the agent is a therapeutic agent.
88. The method according to item 87, wherein the therapeutic agent is selected from the group consisting of analgesics, antipyretics, anesthetics, non-steroidal anti-inflammatoires, antimicrobials, antibiotics, antifungals, antivirals, antiretrovirals, HIV therapies, hypoglycemics, lipid-lowering agents, herbal remedies, traditional Chinese medicine remedies, anticancer agents, antituberculosis agents, and cardiac medications.
89. The method according to item 87 or 88, wherein the therapeutic agent is acetaminophen.
90. The method according to any one of the preceding items, wherein the subject has a pre-existing condition or disease that increases the subject's risk of developing a liver condition or disease when exposed to the agent.
91. The method according to any one of the preceding items, wherein the agent induces oxidative stress in the liver.
92. The method according to item 91, wherein Compound 1 reduces the oxidative stress of the liver of the subject relative to an untreated subject.
93. The method according to item 92, wherein Compound 1 reduces the presence of an oxidative stress biomarker selected from the group consisting of CAT, SOD, NRF2, iNOS, or any combination thereof relative to an untreated subject.
94. The method according to any one of the preceding items, wherein the agent induces necrosis of the liver.
95. The method according to item 94, wherein Compound 1 reduces the necrosis of the liver of the subject relative to an untreated subject.
96. The method according to item 95, wherein Compound 1 reduces the presence of a necrosis biomarker selected from the group consisting of BCL-2, BAX, or a combination thereof relative to an untreated subject.
97. The method according to any one of items 94 to 96, wherein the necrosis of the liver is a central lobular necrosis of the liver.
98. The method according to any one of items 94 to 97, wherein the necrosis of the liver is a focal necrosis of the liver.
99. The method according to any one of the preceding items, wherein the agent induces inflammation of the liver.
100. The method according to item 99, wherein Compound 1 reduces the inflammation of the liver of the subject relative to an untreated subject.
101. The method according to item 100, wherein Compound 1 reduces the presence of an inflammation biomarker selected from the group consisting of p38 MAPK, NF-κB p65, TNF-α, IL-1β, IL-6, and IL-10.
102. The method according to item 101, wherein Compound 1 reduces the presence of an inflammation biomarker selected from the group consisting of TNF-α, IL-1β, and IL-6.
103. The method according to any one of the preceding items, wherein the agent induces an elevation of ALT and AST biomarkers.
104. The method according to item 103, wherein Compound 1 reduces the elevation of ALT and AST biomarkers relative to an untreated subject.
105. The method according to any one of the preceding items, wherein Compound 1 is administered at a dosage of about 0.001 to about 10 mg/kg, about 0.1 to about 10 mg/kg, or about 5 mg/kg to about 10 mg/kg.
106. The method according to any one of the preceding items, wherein the subject is a human subject.
107. The method according to any one of the preceding items, wherein the compound or pharmaceutically acceptable salt thereof is formulated for administration by injection or infusion, preferably subcutaneous, intramuscular, or intravenous injection, or intravenous or subcutaneous infusion.
108. The method according to any of the preceding items, wherein the compound or pharmaceutically acceptable salt thereof is formulated for administration by injection or infusion, preferably intravenous injection or infusion.

The following examples serve to illustrate the invention, however, should not be understood as restricting the scope of the invention.

### EXAMPLES

### Example 1—Acetaminophen-Induced Liver Injury Model in Mice

C57BL/6J wild-type mice were purchased from Guangdong Medical Animal Centre (Guangzhou, China). Male mice (n=8, 8 weeks of age) were randomly assigned to the different treatment groups. Mice were acclimatized to their environment for 1 week before the experiments. Animals were fasted overnight, but allowed access to water, before acetaminophen (APAP) administration.

APAP (Sigma Aldrich) was dissolved in warm phosphate-buffered saline (PBS; 55° C.) that was cooled to 37° C. before injection and administered intraperitoneally with APAP at a dose of 300 mg/kg after 15 hours of starvation. Compound 1 was made fresh for each experiment in sterile 0.9% saline at 20 mg/ml. N-acetylcysteine (NAC, Sigma Aldrich) was made fresh for each experiment in 0.9% saline at 20 mg/ml.

After APAP administration (1 hours), either PBS (Control Group), Compound 1 (10 mg/kg, 5 mg/kg, 2.5 mg/kg, 1 mg/kg) or NAC (150 mg/kg) were injected intravenously in the tail vein of mice. In the Control Group, the mice were injected with phosphate-buffered saline vehicle (labelled “Ctrl” in the Figures), in the Vigilance Group the mice were injected with Compound 1 (labelled “v-Compound 1-10 mg” in the Figures). In both the Control and Vigilance Groups, no APAP was administered.

Each group had 8 mice.

Mice were sacrificed at 6 and 24 hours after administration of APAP. Blood samples and livers were harvested for serum ALT (alanine aminotransferase) and AST (aspartate aminotransferase), and histology. ALT and AST in serum samples were detected using the alanine aminotransferase assay kit (S03030) and the aspartate aminotransferase assay kit (S03040) and automated clinical chemistry analyzer (Chemray 800) from Rayto.

All liver sections were stained with a hematoxylin and eosin (H&E) stain. Necrotic area was measured and expressed as a percentage of necrotic tissue in whole area of liver section. All animal procedures were approved by the Laboratory Animals Ethics Committee of Jinan University (Guangzhou, China).

### Results

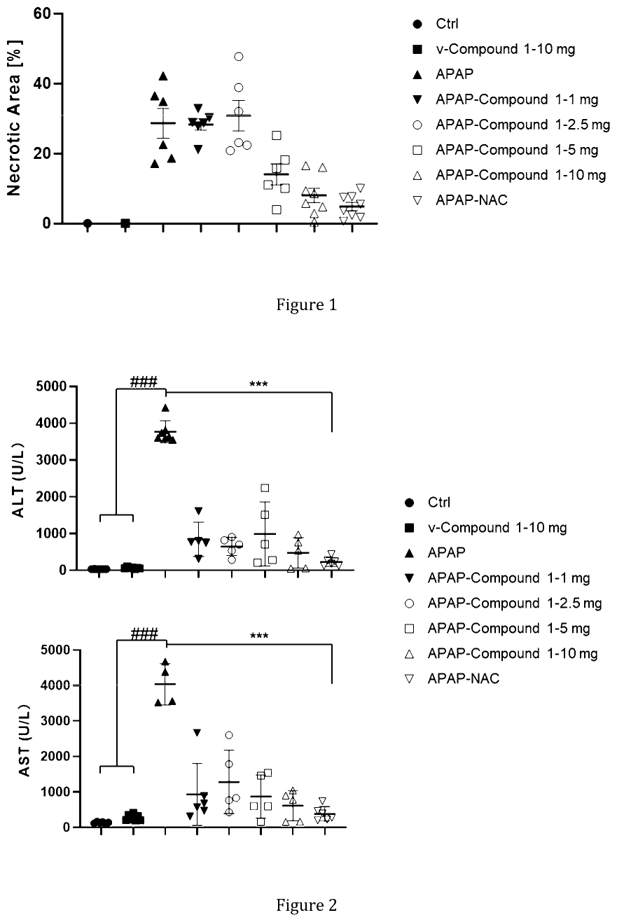

Compound 1 at the 5 mg/kg and 10 mg/kg levels showed liver tissue protection after 6 hours APAP administration as shown in FIG. 1. The 5 mg/kg and 10 mg/kg Compound 1 levels show reduced necrotic area compared to APAP administered alone. Similarly, the ALT and AST biomarker levels are reduced in the presence of 5 mg/kg and 10 mg/kg of Compound 1 after 6 hours APAP administration compared to APAP administered alone as shown in FIG. 2. Moreover, ELISA results after 6 hours APAP administration show reduced inflammation biomarkers (TNF-α, IL-1β, and IL-6) at the 5 mg/kg and 10 mg/kg Compound 1 levels as shown in FIG. 3.

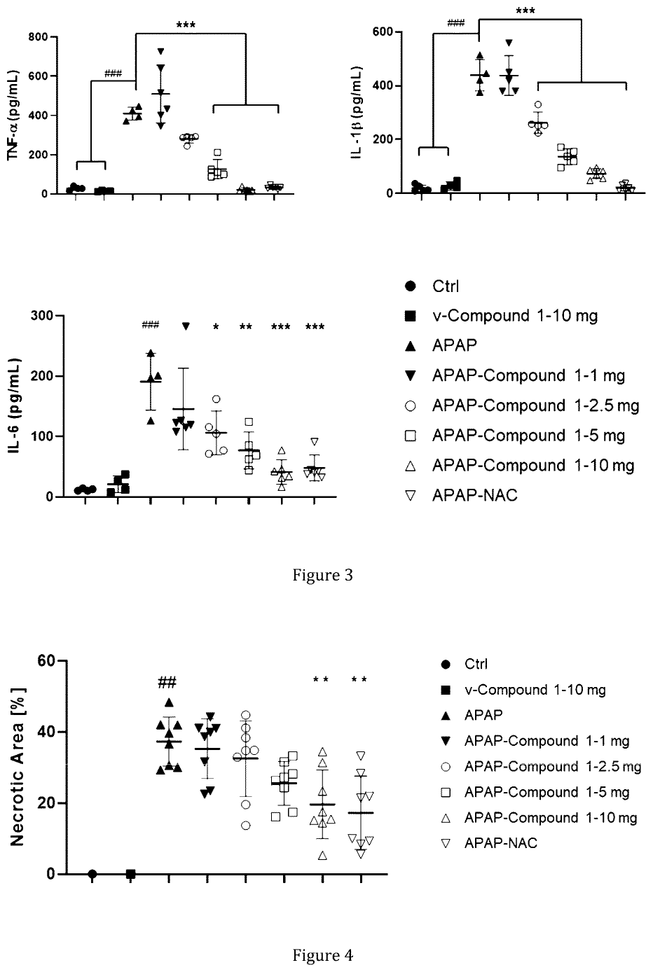

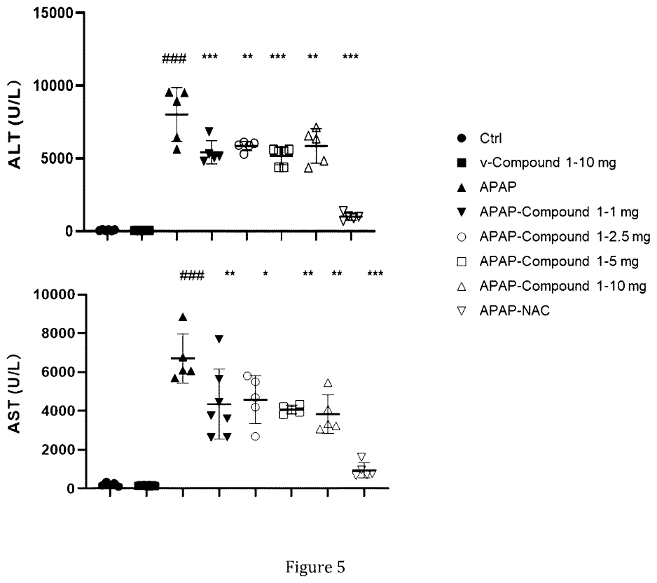

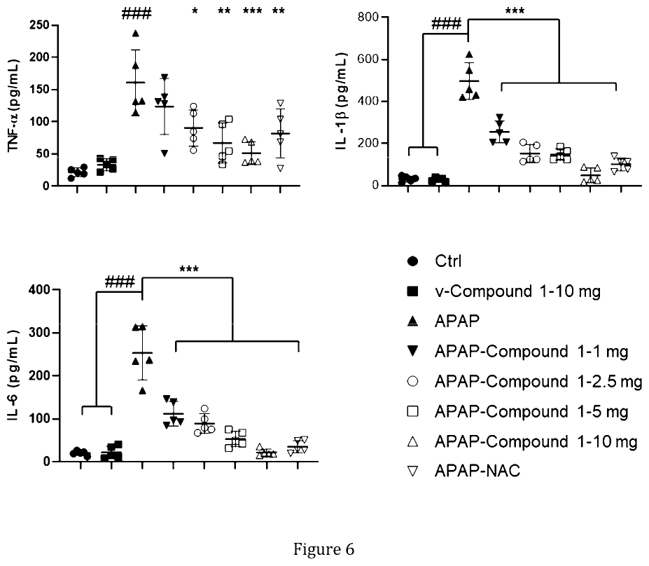

Similar to the results after 6 hours APAP administration, Compound 1 at the 5 mg/kg and 10 mg/kg levels showed liver tissue protection after 24 hours APAP administration as shown in FIG. 4. The 5 mg/kg and 10 mg/kg Compound 1 levels show reduced necrotic area compared to APAP administered alone. Similarly, the ALT and AST biomarker levels are reduced in the presence of 5 mg/kg and 10 mg/kg of Compound 1 after 24 hours APAP administration compared to APAP administered alone as shown in FIG. 5. Finally, ELISA results after 24 hours APAP administration show reduced inflammation biomarkers (TNF-α, IL-1β, and IL-6) at the 5 mg/kg and 10 mg/kg Compound 1 levels as shown in FIG. 6.

### Example 2—Alcohol-Induced Liver Injury in Mice

C57BL/6J wild-type mice were purchased from Guangdong Medical Animal Centre (Guangzhou, China). Male mice (n=7, 8 weeks of age) were randomly assigned to the different treatment groups. The mice were initially fed a controlled Lieber-DeCarli diet ad libitum for 5 days (TP4030C, Trophic Animal Feed High-Tech Co., Ltd, China) to acclimatize them to a liquid diet. Subsequently, the ethanol-fed groups were allowed free access for 8 weeks to an ethanol diet (TP4030D, Trophic) containing 5% (vol/vol) ethanol.

Chronic (8 weeks)-plus-multiple binges (E8w+nB): mice were fed an ethanol Lieber-DeCarli diet for 8 weeks as described above and treated by gavage administration twice a week (5 g/kg ethanol) during the 8-week feeding. Mice were euthanized 9 h post gavage.

The control mice were pair-fed an isocaloric control liquid diet for 8 weeks (P8w), followed by gavage administration of isocaloric liquid dextrin-maltose (Ctrl), without exposure to alcohol.

Each group had 7 mice.

Compound 1 was made fresh for each experiment in sterile 0.9% saline at 20 mg/ml and administered intraperitoneally (10 mg/kg) daily for the last two weeks.

Because mice do not eat after binge, to compare with these groups, E8w mice and the pair-fed mice were subjected to starvation at 7:00 am and sacrificed 9 hours later. Blood samples and livers were harvested for serum ALT and AST, and histology. All liver sections were stained with H&E and Oil Red O staining. The features of alcoholic liver disease (“ALD”) are scored according to the non-alcoholic fatty liver disease (“NAFLD”) activity score (“NAS”) originally developed to measure changes in NAFLD during therapeutic trials but is also applicable to ALD. The ALD score of each group was calculated according to standard procedures.

Oil Red O is a fat-soluble azo dye, used to stain fats, specifically neutral triglycerides, lipids and lipoproteins in tissues and cells. When a tissue section is placed in the dye solution, the dye leaves the dye solution and dissolves in the lipids (such as lipid droplets) in the tissue, staining the lipid droplets in the tissue red. The Oil Red O quantification “a.v.” means a subjective quantity, i.e., “arbitrary value”. The image quantification software ImageJ was used to select the area of oil red staining in liver sections and the area of lipid droplets in quantitative sections.

### Results

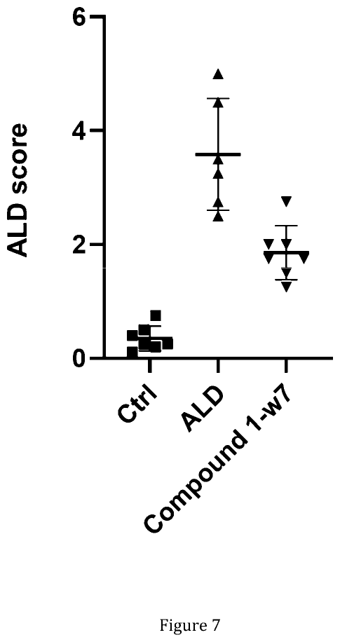

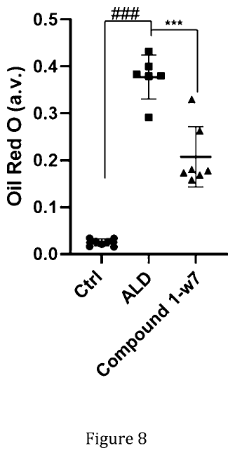

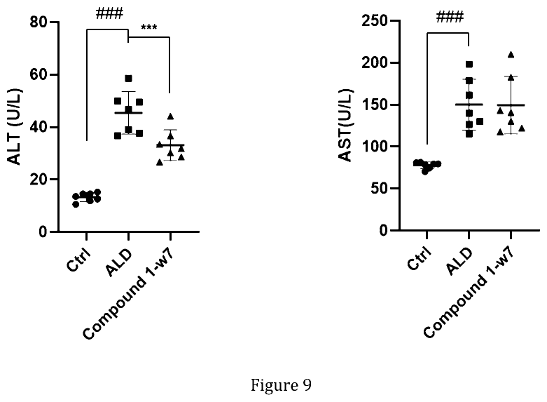

Compound 1 at 10 mg/kg daily dosage for the last two weeks (weeks 7 and 8) reduces the ALD score from about 4 to about 2 compared to untreated E8w+nB mice as shown in FIG. 7. The ALD number is calculated from the weighted sum of hepatocyte fat changes (0 to 3), intralobular inflammation (0 to 3), and hepatocyte balloon-like changes (0 to 2). ALD≥5 may be diagnosed as non-alcoholic steatohepatitis (“NASH”), ALD<3 may exclude NASH, and ALD (3 to 4) indicates NASH is possible. Compound 1 also reduces hepatic lipid accumulation as shown in reduction of Oil Red O liver staining as shown in FIG. 8.

1. Use of Compound 1 or a pharmaceutically acceptable salt thereof 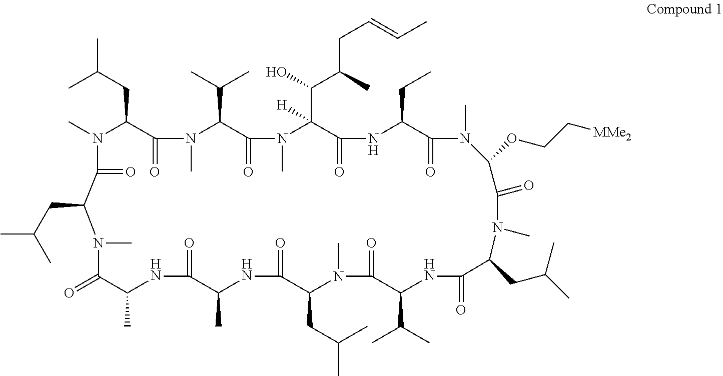
in the manufacture of a medicament for the prevention and/or treatment of a liver condition or disease in a subject exposed to an agent capable of inducing the liver condition or disease.

2. The use according to claim 1, wherein the liver condition or disease is a hepatotoxic condition or disease.

3. The use according to claim 2, wherein the hepatotoxic condition or disease is selected from the group consisting of toxic hepatitis, toxin-induced hepatitis, drug-induced hepatitis, toxin-induced hepatic necrosis, drug-induced hepatic necrosis, toxin-induced hepatic fibrosis, drug-induced hepatic fibrosis, toxin-induced hepatic granuloma, drug-induced hepatic granuloma, toxic liver disease, toxin-induced liver disease, drug-induced liver disease, toxic liver disease with hepatitis, toxin-induced liver disease with hepatitis, drug-induced liver disease with hepatitis, toxin-induced liver damage, drug-induced liver damage, hepatogenous poisoning, toxic liver disease with cholestasis, or a combination thereof.

4. The use according to any one of claims 1 to 3, wherein the liver condition or disease is an acute or chronic liver condition or disease.

5. The use according to any one of claims 1 to 4, wherein the liver condition or disease is an acute liver condition or disease.

6. The use according to any one of claims 1 to 4, wherein the liver condition or disease is a chronic liver condition or disease.

7. The use according to any one of claims 1 to 6, wherein the liver condition or disease is the result of direct toxicity of the agent, immune-mediated mechanisms, or a combination of direct toxicity of the agent and immune-mediated mechanisms.

8. The use according to claim 7, wherein the immune-mediated mechanism is an inflammatory reaction.

9. The use according to any one of claims 1 to 8, wherein the agent is an alcohol.

10. The use according to claim 9, wherein the alcohol is a beverage alcohol.

11. The use according to any one of claims 9 to 10, wherein the alcohol is ethanol.

12. The use according to any one of claims 9 to 11, wherein Compound 1 reduces a non-alcoholic fatty liver disease activity score of the subject relative to an untreated subject.

13. The use according to any one of claims 9 to 12, wherein Compound 1 reduces Oil Red O liver staining of the subject relative to an untreated subject.

14. The use according to any one of claims 1 to 8, wherein the agent is a therapeutic agent.

15. The use according to claim 14, wherein the therapeutic agent is selected from the group consisting of analgesics, antipyretics, anesthetics, non-steroidal anti-inflammatories, antimicrobials, antibiotics, antifungals, antivirals, antiretrovirals, HIV therapies, hypoglycemics, lipid-lowering agents, herbal remedies, traditional Chinese medicine remedies, anticancer agents, antituberculosis agents, and cardiac medications.

16. The use according to claim 14 or 15, wherein the therapeutic agent is acetaminophen.

17. The use according to any one of the preceding claims, wherein the subject has a pre-existing condition or disease that increases the subject's risk of developing a liver condition or disease when exposed to the agent.

18. The use according to any one of the preceding claims, wherein the agent induces oxidative stress in the liver.

19. The use according to claim 18, wherein Compound 1 reduces the oxidative stress of the liver of the subject relative to an untreated subject.

20. The use according to claim 19, wherein Compound 1 reduces the presence of an oxidative stress biomarker selected from the group consisting of CAT, SOD, NRF2, iNOS, or any combination thereof relative to an untreated subject.

21. The use according to any one of the preceding claims, wherein the agent induces necrosis of the liver.

22. The use according to claim 21, wherein Compound 1 reduces the necrosis of the liver of the subject relative to an untreated subject.

23. The use according to claim 22, wherein Compound 1 reduces the presence of a necrosis biomarker selected from the group consisting of BCL-2, BAX, or a combination thereof relative to an untreated subject.

24. The use according to any one of claims 21 to 23, wherein the necrosis of the liver is a central lobular necrosis of the liver.

25. The use according to any one of claims 21 to 24, wherein the necrosis of the liver is a focal necrosis of the liver.

26. The use according to any one of the preceding claims, wherein the agent induces inflammation of the liver.

27. The use according to claim 26, wherein Compound 1 reduces the inflammation of the liver of the subject relative to an untreated subject.

28. The use according to claim 27, wherein Compound 1 reduces the presence of an inflammation biomarker selected from the group consisting of p38 MAPK, NF-κB p65, TNF-α, IL-1β, IL-6, and IL-10.

29. The use according to claim 28, wherein Compound 1 reduces the presence of an inflammation biomarker selected from the group consisting of TNF-α, IL-1β, and IL-6.

30. The use according to any one of the preceding claims, wherein the agent induces an elevation of ALT and AST biomarkers.

31. The use according to claim 30, wherein Compound 1 reduces the elevation of ALT and AST biomarkers relative to an untreated subject.

32. The use according to any one of the preceding claims, wherein Compound 1 is administered at a dosage of about 0.001 to about 10 mg/kg, about 0.1 to about 10 mg/kg, or about 5 mg/kg to about 10 mg/kg.

33. The use according to any one of the preceding claims, wherein the subject is a human subject.

34. The use according to any one of the preceding claims, wherein the medicament is formulated for administration by injection or infusion, preferably subcutaneous, intramuscular, or intravenous injection, or intravenous or subcutaneous infusion.

35. The use according to any of the preceding claims, wherein the medicament is formulated for administration by injection or infusion, preferably intravenous injection or infusion.
